(19) United States
(12) Patent Application Publication
(10) Pub. No.: US 2026/0042730 A1
(43) Pub. Date: Feb. 12, 2026

(54) CATALYTIC CHEMICAL RECYCLING OF POLYAMIDE-BASED PLASTICS

(71) Applicant: Northwestern University, Evanston, IL (US)

(72) Inventors: Yosi Kratish, Evanston, IL (US); Tobin Jay Marks, Evanston, IL (US); Lukas Wursthorn, Konstanz (DE)

(21) Appl. No.: 19/100,825

(22) PCT Filed: Aug. 17, 2023
(86) PCT No.: PCT/US2023/072354

§ 371 Date: Feb. 3, 2025

(51) Int. Cl. C07C 231/14; B01J 31/22; C07C 233/05; C07C 233/06; C08G 69/14; C08G 69/26

(52) U.S. Cl. CPC C07C 231/14; B01J 31/22; C07C 233/05; C07C 233/06; C08G 69/14; C08G 69/26; B01J 2531/37; B01J 2531/38

(57) ABSTRACT

Methods for the solvent-free depolymerization of a polyamide are provided which, in embodiments, comprise combining a polyamide and a lanthanide-organic catalyst comprising a lanthanide metal bound to at least one ligand, to depolymerize the polyamide to a product, wherein the at least one ligand is selected from benzyl and those having a formula -EHₘ(XRₙ)₂. In this formula, E is selected from N, P, C, Si, Ge, and Sn; X is selected from H, N, P, C, Si, Ge, and Sn; R is selected from H, alkyl, aryl, alkoxyl, silyl, germyl, and stannyl; wherein if E is N or P, then m=0 and if E is C, Si, Ge, or Sn, then m=1; and further wherein n is from 2 to 3.

### CROSS-REFERENCE TO RELATED APPLICATIONS

The present application claims priority to U.S. provisional patent application No. 63/399,303 that was filed Aug. 19, 2022, and to U.S. provisional patent application No. 63/466,763 that was filed May 16, 2023, the entire contents of each of which are incorporated herein by reference.

### REFERENCE TO GOVERNMENT RIGHTS

This invention was made with government support under grant number DE-FG02-03ER15457 awarded by the Department of Energy, grant number G-64020-02/DE-SC0022290 awarded by the Department of Energy, and grant number CHE-1856619 awarded by the National Science Foundation. The government has certain rights in the invention.

### BACKGROUND

Modern society depends on plastics-based materials, as evidenced by the growth in their production. This has occurred despite the negative worldwide environmental and health consequences associated with unimaginable amounts of non-degradable waste plastic accumulation and the significant depletion of virgin fossil feedstocks. One class of commonly used plastics for applications in which the material/product must withstand harsh mechanical and environmental conditions are engineering plastics such as polyamides. However, the same properties also impede biodegradation, leading to accumulation in landfills and the environment. The above issues associated with the polyamide Nylon illustrate the need for a circular economy in which waste plastics are recycled and repurposed.

### SUMMARY

Provided are methods for depolymerizing polyamides, e.g., Nylon-6, using lanthanide-organic catalysts which are further described below. Mild conditions may be used along with minimal catalyst loadings to depolymerize the polyamides to products, including monomers from which the polyamides were formed, in high yields. Liquid media and additives are not required. Moreover, the methods are not limited to virgin polyamide powders, but are compatible with post-consumer products. The methods are also selective for the polyamides over other plastics that may be present.

In an embodiment 1, a method for depolymerizing a polyamide comprises combining a polyamide and a lanthanide-organic catalyst comprising a lanthanide metal bound to at least one ligand, to depolymerize the polyamide to a product, wherein the at least one ligand is selected from benzyl and those having a formula -EHₘ(XRₙ)₂. In this formula, E is selected from N, P, C, Si, Ge, and Sn; X is selected from H, N, P, C, Si, Ge, and Sn; R is selected from H, alkyl, aryl, alkoxyl, silyl, germyl, and stannyl; wherein if E is N or P, then in =0 and if E is C, Si, Ge, or Sn, then m=1; and further wherein n is from 2 to 3.

An embodiment 2 is according to embodiment 1, wherein the at least one ligand is selected from those having the formula -EHₘ(XRₙ)₂ and further wherein E is selected from N and C; X is selected from Si, and R is alkyl.

An embodiment 3 is according to embodiment 2, wherein the lanthanide metal is selected from La, Nd, Sm, Gd, Lu, Y, and Sc.

An embodiment 4 is according to any of embodiments 1-3, wherein all ligands bound to the lanthanide metal, including the at least one ligand, are of the same type.

An embodiment 5 is according to embodiment 4, wherein the at least one ligand is selected from those having the formula -EHₘ(XRₙ)₂ and further wherein E is selected from N and C; X is selected from Si, and R is alkyl.

An embodiment 6 is according to embodiment 5, wherein the lanthanide metal is selected from La, Nd, Sm, Gd, Lu, Y, and Sc.

An embodiment 7 is according to any of embodiments 1-6, wherein the lanthanide-organic catalyst is Ln[N(Si(CH₃)₃)₂]₃ or Ln[CH(Si(CH₃)₃)₂]₃, wherein Ln is the lanthanide metal.

An embodiment 8 is according to embodiment 7, wherein Ln is La, Nd, Sm, Gd, Lu, Y, or Sc.

An embodiment 9 is according to embodiment 8, wherein Ln is La or Nd.

An embodiment 10 is according to any of embodiments 1-9, wherein the lanthanide-organic catalyst is La[N(Si(CH₃)₃)₂]₃.

An embodiment 11 is according to any of embodiments 1-10, wherein the polyamide is a polymerization product of a monomer selected from 2-pyrrolidone, 2-piperidone, ε-caprolactam, enantholactam, capryllactam, pelargolactam, azacycloundecan-2-one, azacyclotridecan-2-one, 6-azabicyclo[3.2.1]octan-7-one, and combinations thereof.

An embodiment 12 is according to any of embodiments 1-10, wherein the polyamide is selected from poly(2-pyrrolidinone) (Nylon-4), poly(2-piperidone) (Nylon-5), poly(hexano-6-lactam) (Nylon-6), polyenanthamide (Nylon-7), poly capryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), poly[imino(1,6-dioxo hexamethylene)imino hexamethylene](Nylon-66), and combinations thereof.

An embodiment 13 is according to any of embodiments 1-12, wherein the product comprises a monomer from which the polyamide was formed.

An embodiment 14 is according to embodiment 13, wherein the monomer is a cyclic amide.

An embodiment 15 is according to any of embodiments 1-14, wherein the polyamide and the lanthanide-organic catalyst form a reaction mixture consisting of the polyamide and the lanthanide-organic catalyst.

An embodiment 16 is according to any of embodiments 1-15, wherein the method is carried out using a temperature of 250° C. or less and a loading of the lanthanide-organic catalyst of 5 mol % or less, and the method provides a yield of the product of at least 70%.

An embodiment 17 is according to embodiment 16, wherein the product comprises a monomer from which the polyamide was formed.

An embodiment 18 is according to any of embodiments 1-17, wherein the method is carried out using a continuous flow reactor system.

An embodiment 19 is according to any of embodiments 1-18, wherein the method further comprises recovering the product from a reaction mixture comprising the polyamide and the lanthanide-organic catalyst, wherein the product comprises a monomer from which the polyamide was formed.

An embodiment 20 is according to any of embodiments 1-19, wherein the lanthanide-organic catalyst is Ln[N(Si(CH₃)₃)₂]₃ or Ln[CH(Si(CH₃)₃)₂]₃, wherein Ln is La, Nd, Sm, Gd, Lu, Y, or Sc, and further wherein the method is carried out using a temperature of 250° C. or less and a loading of the lanthanide-organic catalyst of 5 mol % or less.

An embodiment 21 is according to embodiment 20, wherein the polyamide is poly(hexano-6-lactam) (Nylon-6), poly[imino(1,6-dioxo hexamethylene)imino hexamethylene](Nylon-66), or a combination thereof.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1 shows an approach for the chemical recycling of Nylon-6 according to an illustrative embodiment.

FIG. 2 shows yield of caprolactam for different Ln^{NTMS} catalysts vs. their ionic radii. Reaction conditions, 100 mg Nylon-6, 5% catalyst loading, 50 mL Schlenk flask, 240° C., 2 h, 10⁻³ Torr.

FIG. 3A shows the effect of reaction time on ε-caprolactam yield. Conditions: 100 mg Nylon-6, 5% catalyst loading, 50 mL Schlenk flask, 240° C., 10⁻³ Torr FIG. 3B shows the structure of Nylon-6 and binding of the La^{NTMS} catalyst. FIG. 3C shows the effect of catalyst loading on ε-caprolactam yield. Reaction conditions: 100 mg Nylon-6, 50 mL Schlenk flask, 240° C., 45 min, 10⁻³ Torr. Yield determined by ¹H-NMR using mesitylene as internal standard.

FIG. 4 shows the X-ray crystal structure of (TMS₂N)₃La (La^{NTMS}) (hydrogen atoms are omitted for clarity) and experimental and calculated key bond length and angles.

FIGS. 5A-5E show mechanistic DFT analysis of Nylon-6 depolymerization. FIG. 5A shows a computed Nylon-6 model reaction. FIG. 5B, steps (i)-(iii), shows nylon coordination. FIG. 5C, steps (iv-viii), shows a proposed depolymerization mechanism. FIG. 5D shows calculated transition state geometries. FIG. 5E shows computed solution-phase enthalpic profile in kcal/mol.

### DETAILED DESCRIPTION

Provided are methods in which a polyamide and a lanthanide-organic catalyst are combined under conditions to depolymerize the polyamide. The polyamides, lanthanide-organic catalysts, and conditions are described in detail below. The products produced from the depolymerization of the polyamide are also described.

The polyamide to be depolymerized by the present methods is a polymer composed of monomers covalently bound into an extended chain via amide linking groups. The term “monomer” refers to the chemical reactant that is incorporated to form the extended chain and the amide linking groups during a polymerization reaction. The polyamide may be a homopolymer (i.e., formed from a single type of monomer) or a heteropolymer (i.e., formed from more than a one type of monomer, e.g., two). Heteropolymers (which may be referred to as copolymers) may be random or block. The term “type” refers to chemical formula such that a single type means the same chemical formula and different type means different chemical formula. The polyamide may be an aliphatic polyamide (i.e., not comprising aromatic rings) or an aromatic polyamide (i.e., comprising aromatic rings). In embodiments, the polyamide is an aliphatic polyamide.

The polyamide may be one which was formed by ring-opening polymerization of a cyclic amide (i.e., lactam). In such embodiments, the monomer is a cyclic amide and the polymerization reaction is ring-opening polymerization. Illustrative cyclic amides include 2-pyrrolidone, 2-piperidone, ε-caprolactam, enantholactam, capryllactam, pelargolactam, azacycloundecan-2-one, and azacyclotridecan-2-one. As noted below, the polyamide may be one formed from a single type of cyclic amide or multiple, different types (e.g., 2 different types) of cyclic amides.

The polyamide may be one which is formed by condensation of an amine (including a diamine) with an acid (including a diacid). The amine and the acid may be provided on a single chemical compound such as 11-aminoundecanoic acid or ω-aminolauric acid. Alternatively, the amine and the acid may be provided on two different chemical compounds such as hexamethylene diamine and adipic acid. In these embodiments, the monomer is the single chemical compound (with the amine and acid groups) or the two chemical compounds (the dianine and the diacid), and the polymerization reaction is condensation.

The polyamide may be identified by reference to the monomer used to form the polyamide, recognizing that the chemical form of the monomer may be modified by the ensuing polymerization reaction that provides the extended chain and the amide linking groups of the polyamide.

Illustrative polyamides to be depolymerized in the present methods include poly(2-pyrrolidinone) (Nylon-4), poly(2-piperidone) (Nylon-5), poly(hexano-6-lactam) (Nylon-6), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), and poly[imino(1,6-dioxo hexamethylene)imino hexamethylene](Nylon-66). Other illustrative poly amides include copolymer polyamides formed from two or more different types of any of the cyclic amides described above. Other illustrative polyamides include copolymer polyamides formed from one of any of the cyclic amides described above and a strained cyclic amide, e.g., 6-azabicyclo[3.2.1]octan-7-one. Another illustrative polyamide is the copolymer Nylon-6/66. Another illustrative polyamide is the copolymer poly(ε-caprolactone-co-ε-caprolactam).

As noted above, the present methods may be used to depolymerize any of the disclosed polyamides. The depolymerization process deconstructs the polyamide into a product. In embodiments, the depolymerization process provides a monomer (e.g., a cyclic amide) from which the polyamide was formed, i.e., the product comprises the monomer of the polyamide. Throughout this disclosure, the terms “a monomer” and “monomer” encompass both a single type of monomer and multiple, different types of monomers, i.e., depending upon the particular polyamide. In such embodiments, this monomer may be recovered and used to reform the polyamide. This is by contrast to depolymerization processes which do not provide a monomer, but rather a product comprising a chemical compound which cannot reform the polyamide absent additional steps to convert the chemical compound to the monomer. However, the present methods encompass both types of depolymerization processes, i.e., those that provide the monomer of the polyamide and those that provide a different type of product.

In embodiments, the methods depolymerize Nylon-6 to produce ε-caprolactam. In embodiments, the methods depolymerize Nylon-4 to produce 2-pyrrolidone. In embodiments, the methods depolymerize Nylon-5 to produce 2-pyrrolidone. In embodiments, the methods depolymerize Nylon-7 to produce enantholactam. In embodiments, the methods depolymerize Nylon-8 to produce capryllactam. In embodiments, the methods depolymerize Nylon-9 to produce pelargolactam. In embodiments, the methods depolymerize Nylon-10 to produce azacycloundecan-2-one. In embodiments, the methods depolymerize Nylon-11 to produce azacyclotridecan-2-one. In embodiments, the methods depolymerize Nylon-12 to produce w-aminolauric acid. For copolymer polyamides, the methods produce a mixture of the component monomers.

A single type of polyamide or multiple, different types of polyamides may be used in the present methods. The polyamide being depolymerized by the method may comprise or consist of any of the disclosed polyamides or a combination thereof.

The lanthanide-organic catalysts used in the present methods are metal-organic compounds comprising a lanthanide metal and at least one ligand bound to the lanthanide metal. The at least one ligand comprises carbon and hydrogen (thus, provide the lanthanide-organic catalyst), but may further comprise non-carbon and/or non-hydrogen elements, including those described below. In embodiments with multiple ligands, they may be of the same type or different types; however, in embodiments, they are of the same type. In embodiments, there are three ligands which may be of the same type.

The lanthanide metal may be selected from Sc, Y, La, Ce, Pr, Nd, Pm, Sm, Gd, Tb, Dy, Ho, Er, Tm, Yb, and Lu. (In the present disclosure, both Sc and Y may be considered to be lanthanide metals.) In embodiments, the lanthanide metal is La, Nd, Sm, Gd, Lu, Y, or Sc. In embodiments, the lanthanide metal is La or Nd.

The ligands may be selected from those having Formula I: -EHₘ(XRₙ)₂, wherein E is selected from N, P, C, Si, Ge, and Sn; X is selected from H, N, P, C, Si, Ge, and Sn; R is selected from H, alkyl, aryl, alkoxyl, silyl, germyl, and stannyl; wherein if E is N or P, then m=0 and if E is C, Si, Ge, or Sn, then m=1; and further wherein n depends upon the selected X (e.g., n=2 or 3). In Formula I (and all its related formulas, e.g., Formulas IA, IB, etc.), the “—” denotes the covalent bond of the ligand to the lanthanide metal. As noted above, in embodiments, all of the ligands bound to the lanthanide metal are selected from those having Formula I, there may be three such ligands, and they may each be the same.

The ligands may be selected from those having Formula IA: -E(XRₙ)₂, wherein E is selected from N and P; and X, R, and n are as defined above with respect to Formula L In embodiments, the ligands are selected from those having Formula IA1: —N(XRₙ)₂ wherein X, R, and n are as defined above with respect to Formula I. In embodiments, the ligands are selected from those having Formula IA2: —N(SiRₙ)₂ wherein R and n are as defined above with respect to Formula I. In embodiments, the ligands are selected from those having Formula IA3: —N(SiRₙ)₂ wherein R is alkyl and n is 3. In some such embodiments of Formula IA3, the alkyl is linear alkyl, e.g., methyl. As noted above, in embodiments, all of the ligands bound to the lanthanide metal are selected from those having Formula IA, IA1, IA2, or 1A3, there may be three such ligands, and they may each be the same.

The ligands may be selected from those having Formula IB: -EH(XRₙ)₂, wherein E is selected from C, Si, Ge, and Sn; and X, R, and n are as defined above with respect to Formula I. In embodiments, the ligands are selected from those having Formula IB1: —CH(XRₙ)₂ wherein X, R, and n are as defined above with respect to Formula I. In embodiments, the ligands are selected from those having Formula IB2: —CH(SiRₙ)₂ wherein R and n are as defined above with respect to Formula I. In embodiments, the ligands are selected from those having Formula IB3: —CH(SiRₙ)₂ wherein R is alkyl and n is 3. In some such embodiments of Formula IB3, the alkyl is linear alkyl, e.g., methyl. As noted above, in embodiments, all of the ligands bound to the lanthanide metal are selected from those having Formula IB, IB1, IB2, or 1B3, there may be three such ligands, and they may each be the same.

The ligands may be selected from benzyl. The benzyl may be unsubstituted benzyl (i.e., —CH₂—C₅H₆) or substituted benzyl. Substituents on substituted benzyl include alkyl (e.g., methyl) on the benzene ring. Such substituents are useful to increase solubility of the lanthanide-organic catalyst. This includes 3,5-dialkyl (e.g., 3,5-dimethyl) substituents on the benzene ring (the 3,5 positions being meta to the methylene group) and 4-alkyl (e.g., 4-methyl) substituents on the benzene ring (the 4 position being para to the methylene group). In this paragraph, the “—” denotes the covalent bond of the benzyl to the lanthanide metal, although there may be a secondary interaction between the lanthanide metal and the benzene ring. As noted above, in embodiments, all of the ligands bound to the lanthanide metal are selected from benzyl, there may be three such ligands, and they may each be the same.

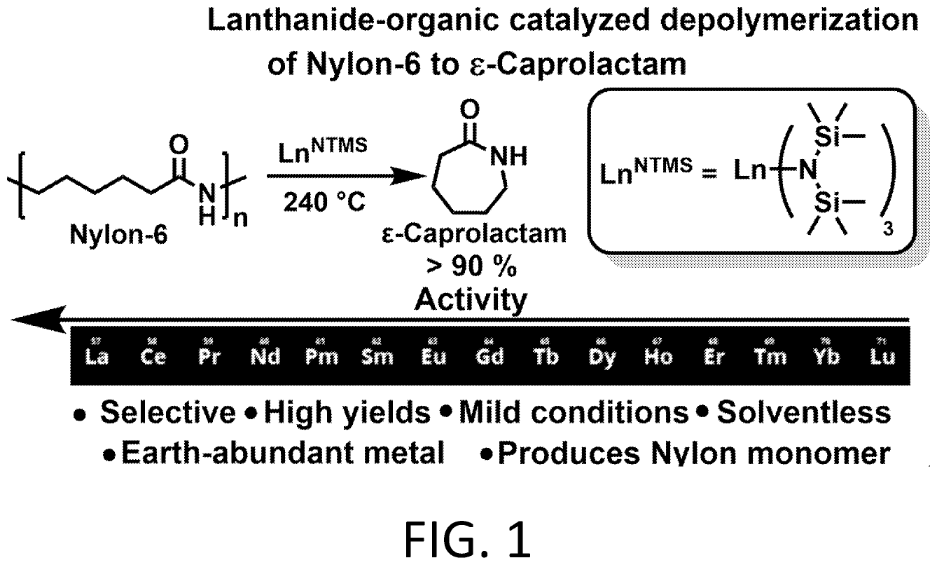

Lanthanide-organic catalysts are encompassed which are based on various combinations of the lanthanide metals and ligands described above, without limitation. However, an illustrative lanthanide-organic catalyst is shown in FIG. 1 having the formula Ln[N(Si(CH₃)₃)₂]₃ (Ln^{NTMS}) wherein Ln is Sc, Y, La, Ce, Pr, Nd, Pm, Sm, Gd, Tb, Dy, Ho, Er, Tm, Yb, or Lu. In embodiments, Ln is La, Nd, Sm, Gd, Lu, Y, or Sc. In embodiments, Ln is La or Nd. In embodiments, the lanthanide-organic catalyst has formula Ln[CH(Si(CH₃)₃)₂]₃ wherein Ln is Sc, Y, La, Ce, Pr, Nd, Pm, Sm, Gd, Tb, Dy, Ho, Er, Tm, Yb, or Lu. In embodiments, Ln is La, Nd, Sm, Gd, Lu, Y, or Sc. In embodiments, Ln is La or Nd.

A single type of lanthanide-organic catalyst or multiple, different types of lanthanide-organic catalysts may be used. The lanthanide-organic catalyst being used to catalyze depolymerization may comprise or consist of any of the disclosed lanthanide-organic catalysts or a combination thereof.

Regarding “alkyl” described herein, this term refers to a linear, branched, or cyclic alkyl in which the number of carbons may range from, e.g., 1 to 8, 1 to 6, 1 to 4, 1 to 3, or 1 to 2. A cyclic alkyl may be referred to as a cycloalkyl. The alkyl may be unsubstituted, by which it is meant the alkyl contains no heteroatoms. An unsubstituted alkyl encompasses an alkyl in which one or more bonds to a carbon(s) or hydrogen(s) are replaced by a bond to an unsubstituted aromatic ring, e.g., benzyl. The alkyl may be substituted, by which it is meant an unsubstituted alkyl in which one or more bonds to a carbon(s) or hydrogen(s) are replaced by a bond to non-hydrogen and non-carbon atoms.

Regarding “aryl” described herein, this term refers to a monocyclic aryl having one aromatic ring (e.g., benzene) or a polycyclic aryl having more than one aromatic ring (e.g., two, three, etc. rings). Monocyclic aryl may be unsubstituted or substituted as described above with respect to alkyl. Regarding polycyclic aryl, neighboring aromatic rings may be fused or unfused. The aromatic rings of a polycyclic aryl may be unsubstituted or substituted as described above with respect to monocyclic aryl.

Regarding “alkoxy” described herein, this term refers to —OR, wherein R is selected from alkyl (as defined herein) and “—” denotes the covalent bond, e.g., to X in Formula I and all its related formulas.

Regarding “silyl” described herein, this term refers to —SiR₃, wherein R is independently selected from H, alkyl, and aryl (each of which has been defined herein) and “—” denotes the covalent bond, e.g., to X in Formula I and all its related formulas.

Regarding “germyl” described herein, this term refers to —GeR₃, wherein R is independently selected from H, alkyl, and aryl (each of which has been defined herein) and “—” denotes the covalent bond, e.g., to X in Formula I and all its related formulas.

Regarding “stannyl” described herein, this term refers to —SnR₃, wherein R is independently selected from H, alkyl, and aryl (each of which has been defined herein) and “—” denotes the covalent bond, e.g., to X in Formula I and all its related formulas.

Regarding substituents in the groups described herein, including those of Formula I and all its related formulas, (as opposed to unsubstituted groups), non-hydrogen and non-carbon atoms include, e.g., halogen; oxygen; sulfur; nitrogen; phosphorus; silicon; germanium; and tin. In embodiments, the group, i.e., alkyl, aryl, alkoxy, silyl, germyl, stannyl, is unsubstituted (which does not preclude the presence of the O in alkoxy, the Si in silyl, the Ge in germyl, and the Sn in stannyl).

The morphology of the lanthanide-organic catalysts may be that of a powder. The particles of the powder may have a size selected to facilitate depolymerization, e.g., a diameter in a range of from 20 Å to 1 mm. This includes from 5 nm to 1 μm, from 10 nm to 500 nm, and from 25 nm to 120 nm.

The conditions being used in the present methods to achieve depolymerization may refer to parameters such as the temperature, atmosphere, time, and lanthanide-organic catalyst loading. As the present methods may be carried out using a variety types of reactor systems, including batch reactor systems, semi-batch reactor systems, and continuous flow reactor systems, the conditions may also refer to a specific type of reactor system. Conditions may be selected in order to facilitate depolymerization, including to achieve a desired (e.g., maximum) yield of product (e.g., monomer). However, as noted above, the present methods are able to achieve high yields even using mild conditions and minimal catalyst loadings.

Regarding temperature, the temperature may be within 30° C., within 20° C., or within 10° C. of the melting temperature of the polyamide. The temperature may be above the melting temperature of the polyamide such that the poly amide is in its molten (liquid) state. The temperature may be in a range of from 100° C. to 350° C. This includes from 100° C. to 300° C., and from 100° C. to 280° C. In embodiments, the temperature is no more than 280° C., no more than 275° C., no more than 265° C., no more than 255° C., or no more than 245° C. This includes a range between any of these values, as well as from 200° C. to 280° C., from 200° C. to 260° C., and from 200° C. to 250° C.

Regarding atmosphere, a vacuum may be used, e.g., 10⁻³ Torr or less, 10⁻⁴ Torr or less, 10⁻⁵ Torr or less, 10⁻⁶ Torr or less. This includes a range between any of these values, as well as from 10⁻³ Torr to 10⁻⁵ Torr. In other embodiments, the method may be carried out under an inert atmosphere (e.g., N₂, dry air, argon) and a pressure in a range of from atmospheric pressure to 10³ Torr.

Regarding time, this may refer to a total period of time over which the polyamide and the lanthanide-organic catalyst are subjected to depolymerization. In embodiments, the period of time is less than 48 hours, less than 36 hours, less than 24 hours, or less than 10 hours. This includes a range between any of these values, as well as from 10 minutes to 10 hours, from 10 minutes to 5 hours, from 1 hour to 10 hours, and from 1 hour to 5 hours. For continuous flow reactor systems, flow rate, rather than time is a relevant parameter. In embodiments, the flow rate is from 5 sccm to 1500 sccm, although higher flow rates may be used. Reactor volume is another relevant parameter for continuous flow reactor systems. In embodiments, the reactor volume is from 50 mL to 1000 L, although greater reactor volumes may be used.

Regarding lanthanide-organic catalyst loading, the loading may be no more than 10 mol %, no more than 8 mol %, or no more than 6 mol %. This includes a range between any of these values, as well as from 0.001 mol % to 5 mol %, 0.1 mol % to 10 mol %, from 0.1 mol % to 8 mol %, from 1 mol % to 8 mol %, and from 2 mol % to 6 mol %. Mol % is calculated based on the of repeating unit of the polyamide. For example, if 1 g of Nylon-6 is used, the number of mol of repeating unit=0.00885 mol (repeating unit of Nylon-6 is 113 g/mol). Thus, the amount of catalyst used to be used is 0.00885 mol*x mol %.

Other conditions that may be used in the present methods include efficient stirring (e.g., from 100 to 4000 rpm), efficient delivery of the polyamide (whether in solid or molten form) to the reaction zone containing the lanthanide-organic catalyst; efficient removal of the product of the depolymerization from the reaction zone, e.g., using heat, low pressure, and flowing gas; and efficient collection of the product, e.g., by cooling of a collection vessel.

The present methods are generally carried out without using any liquid medium, e.g., solvent, for the lanthanide-organic catalyst and the polyamide. As such, the method may be referred to as being “solvent-free,” “solventless,” and the like.

The polyamide (the specific chemical type of which has been described above) may be “virgin” polyamide, which refers to pure, as-synthesized polyamide that not been further processed for use in a particular application (whether the virgin polyamide has been used or not). Alternatively, the polyamide may be “post-consumer” polyamide, which refers to a polyamide which has been further processed to provide a consumer product (whether the consumer product has actually been used or not). Post-consumer polyamide may include other components (e.g., other non-polyamide polymers such as polyolefins, polyesters) and may have been processed for use in a particular application (e.g., yarns, fishing nets, carpet fibers, clothing, medical gloves). In either embodiment, the morphology of the polyamide is not particularly limited. For example, virgin polyamide may be in the form of a powder, including powder composed of micron-sized particles. However, post-consumer polyamide may be in the form of pieces significantly larger than the particles of virgin polyamide powder and in embodiments, no milling, e.g., cryogenic milling, is required to achieve high monomer yields from post-consumer polyamide using the present methods. Regardless of the source or morphology of the polyamide, prior to use in the methods, the polyamide may be washed and dried, e.g., by heating under vacuum.

When combined for carrying out the present methods, the polyamide and the lanthanide-organic catalyst may be considered to form a reaction mixture comprising each of these components. As noted above, the reaction mixture need not comprise any liquid medium (e.g., solvent). (This does not preclude the presence of a liquid in the reaction mixture due to the use of molten polyamide.) Similarly, the reaction mixture need not comprise other additives. Thus, the reaction mixture may be characterized as being free of a liquid medium (other than the polyamide if in its molten state) and free of an additive. This includes the reaction mixture being free of one or more of the following: an ionic liquid; water or steam; ammonia; N,N-dimethylaminopyridine; acetic anhydride; an amine; a phosphoric acid (or salt thereof); a boric acid (or salt thereof); a sulfonic acid (or salt thereof); a carboxylic acid (or salt thereof); a carbonate; an alkali or alkaline earth oxide; an alkali or alkaline earth hydroxide; an alkali or alkaline earth carbonate; an alcohol. In embodiments, the reaction mixture consists of the polyamide and the lanthanide-organic catalyst. The embodiments in this paragraph do not preclude the ultimate presence of the monomer provided by the depolymerization, unreacted polyamide and/or other depolymerized product (e.g., polyamide fragments) in the reaction mixture. These embodiments also do not preclude the presence of components or impurities which may be inherently present in the reaction mixture due to the particular synthetic technique used to form the polyamide (e.g., virgin polyamide). These embodiments also do not preclude the presence of components which may be inherently present in the reaction mixture due to the source of the polyamide (e.g., post-consumer polyamide).

After depolymerization, the present methods may further comprise recovering and/or recycling the lanthanide-organic catalysts from the reaction mixture. No liquid medium is required for recovery of the lanthanide-organic catalysts. The recovered lanthanide-organic catalysts may be used to carry out the method one or more additional times (i.e., they may be recycled).

Similarly, the present methods may further comprise recovering the depolymerized product, e.g., monomer, from the reaction mixture. No liquid medium is required for recovery which may be by vaporization or sublimation. Recovered product may be used for any desired purpose, e.g., recovered monomer may be used to synthesizing a new polymer, including anew polyamide, including the same type of polyamide that was depolymerized.

In addition to the illustrative variations described above, it is to be understood that a variety of reactor types and product collection methods can be employed with the depolymerization catalysts. Reactor types may include but are not limited to: head-over-stirring reactors, batch reactors, rotating flask reactors, horizontal vacuum paddle dryers, and distillation reactors. The product can be collected under vacuum or inert gas flow and condensed in a cold trap or solvent trap. Depolymerization reactions can be operated in batch, semi-batch, or continuous modes of operation.

The present methods may be characterized by a yield of a particular monomer. The yield may be reported as (weight of monomer)/(starting weight of polyamide)*100%. The yield may be determined using ¹H NMR as described in the Example below. The yield may be an initial yield obtained by using fresh (i.e., unused) lanthanide-organic catalyst. The initial yield may be at least 70%, at least 75%, at least 80%, at least 85%, at least 90%, at least 95%, or at least 98%. This includes a range between any of these values, as well as from 70% to 100%, from 80% to 100%, from 80% to 95%. The yield may be a yield obtained by using recovered lanthanide-organic catalyst which has been used one or more times (e.g., 1, 2, 3, 4, etc.). The yield from a recycled/recovered lanthanide-organic catalyst may be within ±20%, ±10%, ±5%, or +2% of the initial yield. Any of the yield values in this paragraph may refer to a specific polyamide (e.g., Nylon-6), a specific monomer (e.g., ε-caprolactam), a specific lanthanide-organic catalyst (e.g., La^{NTMS}) and the method carried out under a specific set of conditions (e.g., batch reactor, temperature (e.g., 240° C.), under vacuum or an inert atmosphere of 1 atm, time (e.g., 2 hours), and loading (e.g., 5 mol %).

As noted above, the polyamide to be depolymerized by the present methods may be provided in a blend with non-polyamide polymers, e.g., a polyolefin (e.g., polyethylene, polypropylene) or a polyester (e.g., polyethylene terephthalate). The method may depolymerize the polyamide while leaving the other polymer intact. The term “intact” does not necessarily mean perfectly intact as a minor amount of the non-polyamide polymer may be decomposed. Thus, the present methods allow for separation of a polyamide from such a blend.

The present disclosure encompasses any of the lanthanide-organic catalysts described herein as well as reaction mixtures comprising (or consisting of) such catalysts and any of the disclosed polyamides.

### EXAMPLE

Additional data, figures, and information, including that referenced as “not shown” below, may be found in U.S. Application No. 63/399,303, filed Aug. 19, 2022, which is hereby incorporated by reference in its entirety.

### Introduction

This Example shows that tris[bis(trimethylsilyl)amido]lanthanide complexes (Ln^{NTMS}) efficiently catalyze the depolymerization of Nylon-6 to ε-caprolactam (FIG. 1). This reaction is clean (>95% selectivity to ε-caprolactam), has high isolated yield (>90%), is solventless, and proceeds at a very low depolymerization temperature, 240° C. (Eq. 1). The catalytic activity scales with the lanthanide ion size, with the largest ion (La³⁺) exhibiting the highest activity. The reaction is effective for virgin polymer and post-consumer products and is also compatible with admixed plastics such as polyethylene (PE), polypropylene (PP), and polyethylene terephthalate (PET). Kinetic investigations and DFT calculations suggest a chain-end back-biting mechanism (vide infra).

### EXPERIMENTAL

### Materials and Methods

All manipulations of reagents were carried out in oven-dried glass reaction vessels. Depolymerization reactions were carried out in cylindrical 50 mL Schlenk tubes with heating supplied by an oil bath for T≤240° C. or a metal bead bath for T>240° C. Nylon-6 powder with a mean particle size of 15-20 μm and a molecular weight of 11930 g/mol (as determined by GPC) was obtained from Goodfellow Inc. LaOTf₃, La^{NTMS}, and Sm^{NTMS} were obtained from Sigma Aldrich, Gd^{NTMS} was obtained from Thermo Scientific, La(N(Tf)₂)₃ was obtained from TCI Chemicals, LaCl₃ was obtained from Alfa Aesar, and La(OⁱPr)₃ was obtained from Acros Organics. Nylon-6 yarn was acquired from The Singer Company. Catalysts were used as received without further purifications. All polymers were dried under a high vacuum or in a vacuum oven for at least 24 h prior to use.

### Physical and Analytical Methods

NMR spectra were recorded on a Varian Bruker Avance III HD system equipped with a TXO Prodigy probe (500 MHz) spectrometer. Chemical shifts (δ) for ¹H-NMR were referenced to the internal solvent. TGA experiments were conducted on a TGA 4000 thermal gravimetric analyzer (PerkinElmer, USA) using the software Pyris. The instrument was operated inside a glovebox to provide an inert atmosphere. Argon was used as a purge gas at a flow rate of 20 mL/min. Nylon-6 yarn was ground in a cryogenic grinder (6775 Freezer/Mill by SPEX Sample Prep). Gel permeation chromatography (GPC) was used to analyze molecular weight and molecular weight dispersity indices of Nylon-6 starting material and depolymerization timepoint samples. An Agilent Infinity 111260 high-performance liquid chromatography (HPLC) system was coupled with Wyatt MiniDAWN TREOS Multi-Angle Light Scattering (MALS, 3 angles) and Refractive Index (RI, 658 nm) detectors. Polymer concentrations of ˜5.0 mg/ml in hexafluoroisopropanol (HFIP, ChemImpex) were prepared by dissolving polymer at 30° C. in a shaker, followed by filtration through a 0.2 μm syringe filter. Samples were injected at a volume of 100 μL into the system at a flow rate of 0.4 mL/min (HFIP with 20 mM NaTFA) through the column at 40° C., which consisted of 3 Plgel-HFIP columns in series with a guard column. Astra software was used to determine absolute Mₙ, M_{w}, and dispersity (Ð, M_{w}/Mₙ) using a dn/dc value of 0.241 mL/g (value found on American Polymer Standard Corporation website for nylon-6,6 in HFIP with 10 mM NaTFA). Polymethyl methacrylate and poly(ethylene terephthalate) standards were used to check instrumentation and validate results. For samples that showed a major peak at earlier retention times and a set of oligomer peaks at later times, the major peak was analyzed separately from the minor set.

### General Depolymerization Procedure

General Procedure for depolymerization reactions. In a glove box, an oven-dried Schlenk tube was charged with a magnetic stir bar, Nylon-6 powder, and finely ground catalyst. The vessel was sealed tightly, and the powders were thoroughly mixed by gently shaking the reaction vessel. The Schlenk tube was then evacuated to 10⁻³ Torr, sealed, and heated to 240° C. with slow magnetic stirring (50 rpm) for the specified time. Occasionally magnetic stirring slowed or stopped. Gently tapping the reaction tube for the first 2-3 mins helped prevent clumping. During the reaction, the products sublimed from the hot reaction zone and deposited as a crystalline layer on the cold wall of the reactor. After cooling to room temperature, the soluble part of the reaction mixture was dissolved in 1-2 mL of CDCl₃, and mesitylene was added as an internal standard. A sample of this solution was withdrawn for NMR analysis. Yields were determined by ¹H-NMR, comparing the signal intensities of ε-caprolactam and mesitylene.

NMR spectra showed the signals of the deuterated solvent CDCl₃, the internal standard mesitylene, the signals of ε-caprolactam, cyclic dimer/oligomers, open-chain amide, and hexamethyldisilazane which is the protonolysis product the Ln^{NTMS} catalysts. (See U.S. Application No. 63/399,303 for the spectra.) Trace amounts of ε-caprolactam, its dimer/oligomers, and the open chain amide were already present in the commercial Nylon-6 starting material, as evident by the ¹H NMR spectrum of its CDCl₃ extract.

### Procedures of Reactions in Tables 1 and 2

TABLE 1
Entry 1

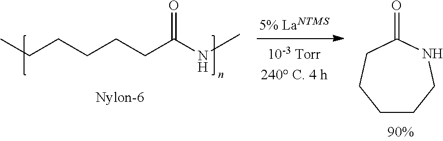

Exactly 102 mg of Nylon-6 (0.90 mmol) and 27.6 mg of La^{NTMS} (45 μmol, 5 mol %) were reacted according to the general procedure, heating the reaction mixture to 240° C. for 4 h. Exactly 22.4 mg of mesitylene (0.186 mmol) was used as an internal standard. ε-caprolactam was obtained in 90% yield.

TABLE 1
Entry 2

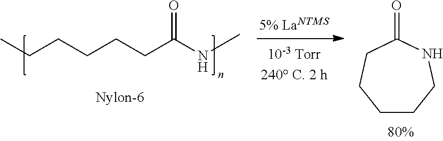

Exactly 98 mg of Nylon-6 (0.87 mmol) and 28.0 mg of La^{NTMS} (45 μmol, 5 mol %) were reacted according to the general procedure, heating the reaction mixture to 240° C. for 2 h. Exactly 17.7 mg of mesitylene (0.147 mmol) was used as internal standard. ε-caprolactam was obtained in 80% yield.

TABLE 1
Entry 3

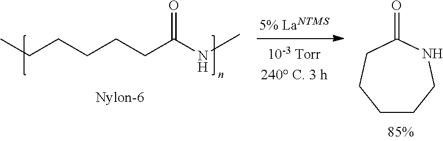

Exactly 99 mg of Nylon-6 (0.87 mmol) and 28.0 mg of La^{NTMS} (45 μmol, 5 mol %) were reacted according to the general procedure, heating the reaction mixture to 240° C. for 3 h. Exactly 18.1 mg of mesitylene (0.151 mmol) was used as internal standard. ε-caprolactam was obtained in 85% yield.

TABLE 1
Entry 4

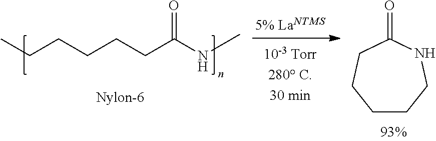

Exactly 102 mg of Nylon-6 (0.90 mmol) and 27.6 mg of La^{NTMS} (45 μmol, 5 mol %) were reacted according to the general procedure, heating the reaction mixture to 280° C. for 30 min. Exactly 17.7 mg of mesitylene (0.147 mmol) was used as internal standard. ε-caprolactam was obtained in 93% yield.

TABLE 1
Entry 5

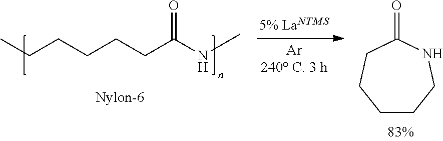

Exactly 100 mg of Nylon-6 (0.88 mmol) and 27.0 mg of La^{NTMS} (43 μmol, 5 mol %) were reacted under inert atmosphere, according to the general procedure without evacuating the reaction vessel, heating the reaction mixture to 240° C. for 3 h. Exactly 18.0 mg of mesitylene (0.150 mmol) was used as internal standard. ε-caprolactam was obtained in 83% yield.

TABLE 1
Entry 6

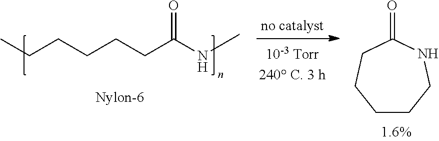

Exactly 100 mg of Nylon-6 (0.88 mmol) was added to a Schlenk flask and evacuated to 10⁻³ Torr. The polymer was heated to 240° C. for 3 h. Exactly 15.0 mg of mesitylene (0.125 mmol) was used as internal standard. ε-caprolactam was obtained in 1.6% yield.

TABLE 1
Entry 7

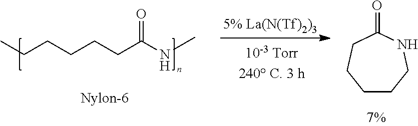

Exactly 100 mg of Nylon-6 (0.88 mmol) and 43.0 mg of La(N(Tf)₂)₃ (44 μmol, 5 mol %) were reacted according to the general procedure, heating the reaction mixture to 240° C. for 3 h. Exactly 18.3 mg of mesitylene (0.152 mmol) was used as internal standard. ε-caprolactam was obtained in 7% yield.

TABLE 1
Entry 8

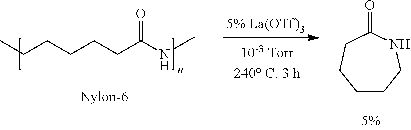

Exactly 99 mg of Nylon-6 (0.87 mmol) and 26.9 mg of La(OTf)₃ (43 μmol, 5 mol %) were reacted according to the general procedure, heating the reaction mixture to 240° C. for 3 h. Exactly 18.9 mg of mesitylene (0.157 mmol) was used as internal standard. ε-caprolactam was obtained in 5% yield.

TABLE 1
Entry 9

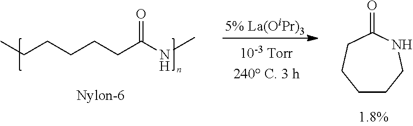

Exactly 99 mg of Nylon-6 (0.87 mmol) and 12.5 mg of La(OⁱPr)₃ (40 μmol, 5 mol %) were reacted according to the general procedure, heating the reaction mixture to 240° C. for 3 h. Exactly 9.2 mg of mesitylene (77 μmol) was used as internal standard. ε-caprolactam was obtained in 1.8% yield.

TABLE 1
Entry 10

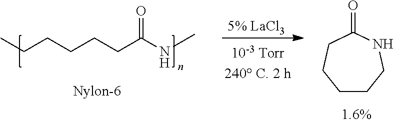

Exactly 101 mg of Nylon-6 (0.89 mmol) and 11.0 mg of LaCl₃ (45 mol, 5 mol %) were reacted according to the general procedure, heating the reaction mixture to 240° C. for 3 h. Exactly 22.5 mg of mesitylene (0.187 mmol) was used as internal standard. ε-caprolactam was obtained in 1.6% yield.

TABLE 1
Entry 11

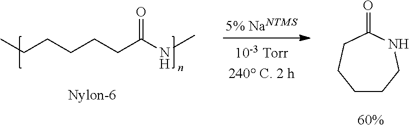

Exactly 101 mg of Nylon-6 (0.89 mmol) and 27.6 mg of Nd^{NTMS} (44 μmol, 5 mol %) were reacted according to the general procedure, heating the reaction mixture to 240° C. for 2 h. Exactly 17.1 mg of mesitylene (0.142 mmol) was used as internal standard. ε-caprolactam was obtained in 60% yield.

TABLE 1
Entry 12

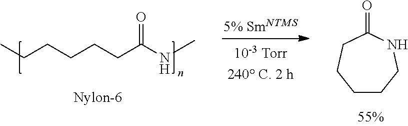

Exactly 99.5 mg of Nylon-6 (0.88 mmol) and 28.0 mg of Sm^{N}MS (44 mol, 5 mol %) were reacted according to the general procedure, heating the reaction mixture to 240° C. for 2 h. Exactly 18.7 mg of mesitylene (0.156 mmol) was used as internal standard. ε-caprolactam was obtained in 55% yield.

TABLE 1
Entry 13

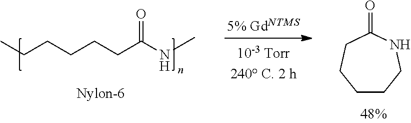

Exactly 101 mg of Nylon-6 (0.89 mmol) and 29.3 mg of Gd^{NTMS} (46 μmol, 5 mol %) were reacted according to the general procedure, heating the reaction mixture to 240° C. for 2 h. Exactly 20.7 mg of mesitylene (0.172 mmol) was used as internal standard. ε-caprolactam was obtained in 48% yield.

TABLE 1
Entry 14

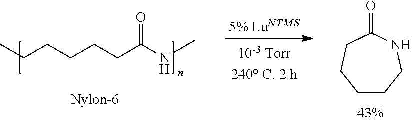

Exactly 100.5 mg of Nylon-6 (0.87 mmol) and 27.6 mg of Lu^{NTMS} (42 μmol, 5 mol %) were reacted according to the general procedure, heating the reaction mixture to 240° C. for 2 h. Exactly 17.4 mg of mesitylene (0.145 mmol) was used as internal standard. F-caprolactam was obtained in 43% yield.

TABLE 1
Entry 15

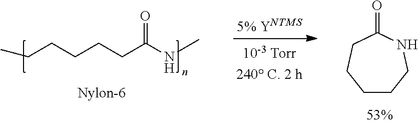

Exactly 101 mg of Nylon-6 (0.89 mmol) and 25.5 mg of Y^{NTMS} (45 μmol, 5 mol %) were reacted according to the general procedure, heating the reaction mixture to 240° C. for 2 h. Exactly 18.9 mg of mesitylene (0.157 mmol) was used as internal standard. ε-caprolactam was obtained in 53% yield.

TABLE 1
Entry 16

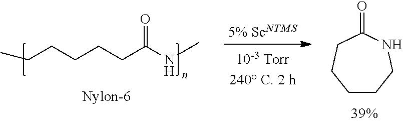

Exactly 85.2 mg of Nylon-6 (0.75 mmol) and 19.4 mg of Sc^{NTMS} (37 μmol, 5 mol %) were reacted according to the general procedure, heating the reaction mixture to 240° C. for 2 h. Exactly 19.9 mg of mesitylene (0.166 mmol) was used as internal standard. ε-caprolactam was obtained in 39% yield.

TABLE 2
Entry 1

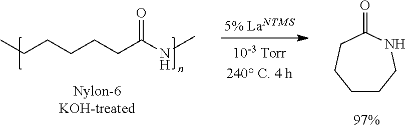

In a scintillation vial, 1.0 g of Nylon-6 was suspended in 3 mL 1M KOH for 11 d, occasionally shaking the vial. The Nylon powder was then collected by filtration and washed with 50 mL of deionized water. The powder was next resuspended in deionized water for another 3 d. The powder was collected by filtration, washed with another 50 mL of deionized water, and dried in a vacuum oven overnight. Exactly 96.5 mg of this base-treated Nylon-6 (0.85 mmol) and 5.4 mg of La^{NTMS} (8.7 μmol, 1.0 mol %) were reacted according to the general procedure, heating the reaction mixture to 240° C. for 4 h. Exactly 18.6 mg of mesitylene (0.155 mmol) was used as an internal standard. ε-caprolactam was obtained in 97% yield.

TABLE 2
Entry 2

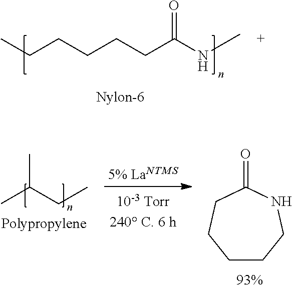

In a glove box, an oven-dried Schlenk tube was charged with a magnetic stir bar, 102 mg of Nylon-6 powder (0.90 mmol), 28.4 mg of La^{NTMS} (46 mol, 5 mol %), and 102 mg of polypropylene. The vessel was sealed tightly and the powders were thoroughly mixed by gently shaking the reaction vessel. The Schlenk tube was then evacuated to 10⁻³ Torr and heated to 240° C. while magnetic stirring for 6 h. After cooling to room temperature, the soluble part of the reaction mixture was dissolved in 1-2 mL of CDCl₃ and 17.8 mg of mesitylene (0.148 mmol) was added as internal standard. ε-caprolactam was obtained in 93% yield. The CDCl₃ insoluble part was treated with 5 mL of trifluoroethanol to dissolve any unreacted Nylon. The leftover Polypropylene was dissolved in CD₂Cl₄ and investigated by ¹H and ¹³C NMR, demonstrating that the polymer was unaffected.

TABLE 2
Entry 3

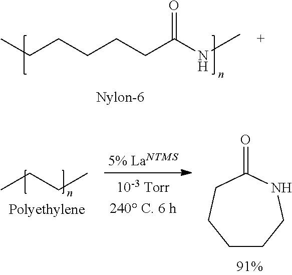

In a glove box, an oven-dried Schlenk tube was charged with a magnetic stir bar, 98 mg of Nylon-6 powder (0.87 mmol), 28.2 mg of La^{NTMS} (45 μmol, 5 mol %), and 101 mg of polyethylene. The tube was sealed tightly, and the powders were thoroughly mixed by gently shaking the reaction vessel. The Schlenk tube was then evacuated to 10⁻³ Torr and heated to 240° C. while magnetic stirring for 6 h. After cooling to room temperature, the soluble part of the reaction mixture was dissolved in 1-2 mL of CDCl₃ and 17.2 mg of mesitylene (0.143 mmol) was added as internal standard. ε-caprolactam was obtained in 91% yield. The CDCl₃ insoluble part was treated with 5 mL of trifluoroethanol to dissolve any unreacted Nylon. The leftover Polyethylene was dissolved in CD₂Cl₄ and investigated by ¹H and ¹³C NMR, demonstrating that the polymer was unaffected.

TABLE 2
Entry 4

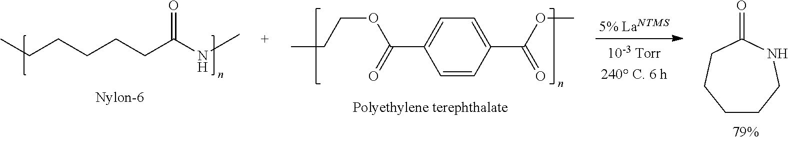

In a glove box, an oven-dried Schlenk tube was charged with a magnetic stir bar, 103 mg of Nylon-6 powder (0.91 mmol), 28.0 mg of La^{NTMS} (45 μmol, 5 mol %), and 115 mg of polyethylene terephthalate. The tube was sealed tightly, and the powders were thoroughly mixed by gently shaking the reaction vessel. The Schlenk tube was then evacuated to 10⁻³ Torr and heated to 240° C. while magnetic stirring for 6 h. After cooling to room temperature, the soluble part of the reaction mixture was dissolved in 1-2 mL of CDCl₃, and 22.6 mg of mesitylene (0.188 mmol) was added as an internal standard. ε-caprolactam was obtained in 77% yield. The CDCl₃ insoluble part was treated with 5 mL of hexafluoroisopropanol to dissolve any unreacted Nylon as well as the polyethylene terephthalate.

TABLE 2
Entry 5

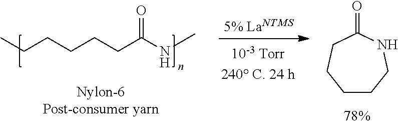

Nylon-6 yarn was obtained from the Singer Company. The yarn was cut into small pieces and then ground in a cryogenic grinder to increase the surface area. The material was dried in a vacuum oven overnight and transferred to a glove box. An oven-dried Schlenk tube was charged with a magnetic stir bar, 113 mg of Nylon-6 yarn (1.00 mmol) and 27.4 mg of La^{NTMS} (44 μmol, 4 mol %). The tube was sealed tightly, and the powders were thoroughly mixed by gently shaking the reaction vessel. The reaction mixture was heated to 240° C. while magnetic stirring for 24 h. After cooling to room temperature, the soluble part of the reaction mixture was dissolved in 1-2 mL of CDCl₃ and 18.2 mg of mesitylene (0.151 mmol) was added as internal standard. ε-caprolactam was obtained in 78% yield.

### Reaction Time and Catalyst Loading Dependence

To investigate the reaction order in respect to Nylon-6, a series of depolymerization reactions at different reaction times was conducted under otherwise identical conditions:

Exactly 100 mg (0.89 mmol) of Nylon-6 and 27.5 mg of La^{NTMS} (44 mol, 5 mol %) were reacted according to the general procedure, heating the reaction mixture to 240° C. for the timeframe listed in Table A. After cooling to room temperature, the reaction mixture was dissolved in CDCl₃ and mesitylene was added as an internal standard for ¹H-NMR. The yields were determined by comparing the ¹H-NMR signal integrals of ε-Caprolactam and mesitylene.

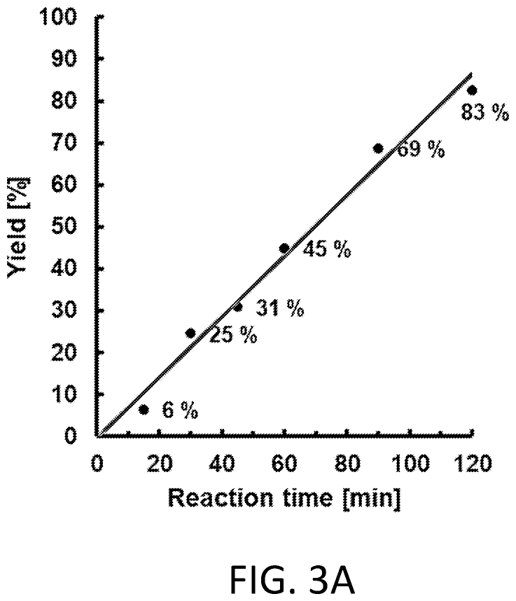

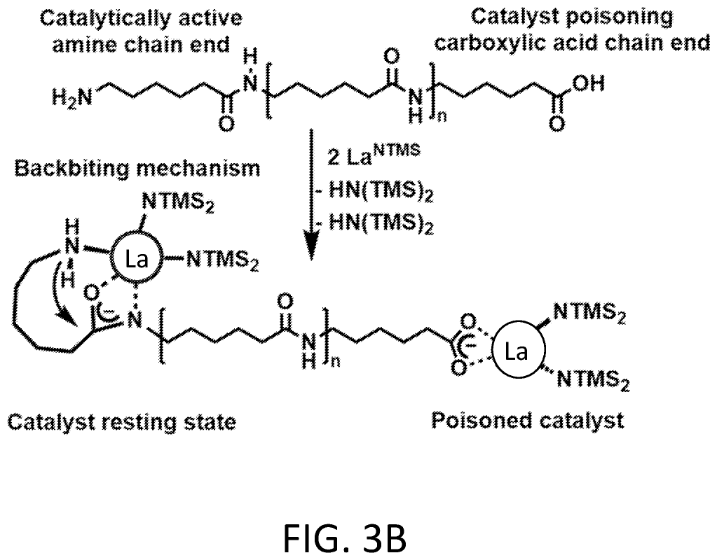

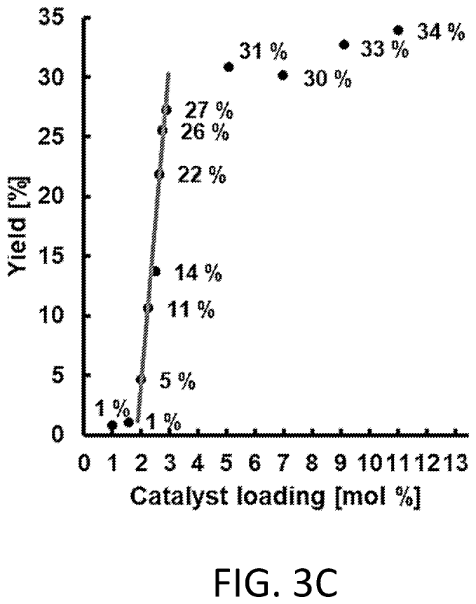

TABLE A
ε-Caprolactam yields at different reaction times (data
points for FIG. 3A). Conditions: 100 mg Nylon-6, 5% catalyst
loading, 50 mL Schlenk flask, 240° C., 10⁻³ Torr.

| Reaction time | ε-Caprolactam yield |
|---|---|
| 15 min | 6% |
| 30 min | 25% |
| 45 min | 31% |
| 60 min | 45% |
| 90 min | 69% |
| 120 min | 83% |

Similarly, to determine the reaction order in respect to La^{NTMS} catalyst, a series of depolymerization reactions at different catalyst loadings was conducted under otherwise identical conditions:

Exactly 100 mg (0.89 mmol) of Nylon-6 and the respective loading of La^{NTMS} (Table B) were reacted according to the general procedure, heating the reaction mixture to 240° C. for 45 min. After cooling to room temperature, the reaction mixture was dissolved in CDCl₃ and mesitylene was added as internal standard for ¹H-NMR. The yields in Table B were determined by comparing the ¹H-NMR signal integrals of ε-caprolactam and mesitylene.

TABLE B
ε-Caprolactam yields at different catalyst loadings
(data points for FIG. 3C). Conditions: 100 mg Nylon-6, 50
mL Schlenk flask, 240° C., 10⁻³ Torr, 45 min.

| Catalyst loading [mol %] | ε-Caprolactam yield |
|---|---|
| 1.00% | 1% |
| 1.57% | 1% |
| 2.01% | 5% |
| 2.25% | 11% |
| 2.49% | 14% |
| 2.65% | 22% |
| 2.74% | 26% |
| 2.88% | 27% |
| 5.08% | 31% |
| 6.97% | 30% |
| 9.11% | 33% |
| 11.02% | 34% |

### Gel Permeation Chromatography (GPC) Analysis of the Residual Nylon-6 Solid

To improve the accuracy of the GPC analysis, the source of the starting Nylon-6 (powder from Goodfellow, M_{w}=12 kDa) was changed to a starting material that exhibited a more uniform distribution (Goodfellows Nylon-6 film, cryomilled and sieved to a 180 μm particle size, M_{w}=36.4 kDa).

TABLE C
Gel permeation chromatography (GPC) analysis of the residual
Nylon-6 solid at different times. Conditions: 100 mg Nylon-6,
10% catalyst loading, 50 mL Schlenk flask, 260° C., Ar.

| Timepoint | Mₙ (kDa) | M_{w} (kDa) | Ð | ε-Caprolactam yield |
|---|---|---|---|---|
| 0 | 25.4 | 36.4 | 1.43 | — |
| 0.5 h | 10.8 | 20.2 | 1.87 | 17.2 |
| 1 h | 6.81 | 11.9 | 1.75 | 35.0 |
| 1.5 h | 5.90 | 12.4 | 2.11 | 43.5 |
| 2 h | 4.30 | 8.18 | 1.90 | 55.7 |
| 4 h | 6.01 | 8.33 | 1.39 | 89.9 |

Kinetic Data from TGA Experiments

Exactly 246 mg of Nylon-6 (2.17 mmol) and 136 mg of La^{NTMS} (0.219 mmol, 10 mol %) were mixed and ground to a fine powder in a mortar and pestle. Samples of approximately 10 mg were heated at 10, 15, 20, and 30° C./min in the temperature range of 30 to 650° C. (Raw data not shown.)

Mass loss of the difference was calculated from the raw TGA data:

$$Mass loss ⌊ \% ⌋ = 100 * \frac{m ( {T}_{start} ) - m ( {T}_{e n d} )}{{m}_{0}}$$

The employed catalyst/polymer mixture consists of 64.4 wt % Nylon-6 and 35.6 wt % La^{NTMS} catalyst. The catalyst contribution can be further broken down into the lanthanum(III) ion center (8.0 wt %) and the —NTMS ligand (9.2 wt % per ligand, 27.6 wt % total).

Activation Energies were determined using isoconversional analysis by the Flynn-Wall-Ozawa (FWO) method. There are several stages of mass loss in the observed temperature range, namely Catalyst activation and sublimation below 270° C., catalytic depolymerization starting around 270° C., and pyrolysis starting around 350° C. The catalyst activation process should be disregarded in the determination of kinetic parameters since it is an inherently different process from depolymerization. Removal of preceding mass loss is a common way to deal with water loss and other volatiles before the observed reaction commences. Since the first mass loss was complete at a temperature of 270° C., this point was chosen as the endpoint of the first mass loss and as the starting point (T₀ and m₀) for calculating conversions and kinetic parameters. Conversions were calculated from the measured masses using the general formula in equation (1).

$$Conversion α ( T ) = \frac{{m}_{0} - m ( T )}{{m}_{0} - {m}_{f}} \qquad ( 1 )$$

Using the basic Arrhenius equation and the heating rate

$$β = \frac{d T}{dt} ,$$

one can derive equation (2), which is the basis for most approaches to extracting kinetic data from TGA experiments.

$$\frac{d α}{dT} = \frac{A}{β} * exp ( - \frac{E}{R T} ) * f ( α ) \qquad ( 2 )$$

For the determination of activation energies, the Flynn-Wall-Ozawa method was chosen. Integration of equation (2) taking the logarithm and using the Doyle approximation gives the linear equation (3):

$$ln ( β ) = 5.3305 + ln ( \frac{A}{F ( α )} ) - 1.052 * \frac{E}{R {T}_{α}} with F ( α ) = {\int }_{0}^{α} \frac{1}{f ( α )} d α \qquad ( 3 )$$

By plotting ln(β) vs. 1/T and measuring the slope, activation energies can be calculated as described in equation 4:

$$\begin{matrix}\frac{d ln ( β )}{d \frac{1}{T}} = - 1.052 * \frac{E}{R {T}_{α}} & E = - \frac{d ln ( β )}{d \frac{1}{T}} * R / 1.052\end{matrix} \qquad ( 4 )$$

The investigated temperature range was 288° C. to 326° C., well below the temperatures required for (uncatalyzed) pyrolysis of Nylon-6, which starts around 350° C. Using this approach, activation energies were calculated at four different conversions (8%, 12%, 16%, and 20%) and averaged. The errors reported for activation parameters were determined using the Regression Analysis workbook in Excel (R. Carr, Deakin University. Australia and Neville Hunt, Coventry University, UK, as part of the OATBRAN project).

TABLE D
Activation energies from isoconversional TGA
obtained using the Flynn-Wall-Ozawa method.

| | | Activation | Standard |
|---|---|---|---|
| Entry | Conversion | energy | Deviation |
| 1 | 8% | 37.5 kcal | 7.9 kcal |
| 2 | 12% | 32.8 kcal | 6.4 kcal |
| 3 | 16% | 30.0 kcal | 5.7 kcal |
| 4 | 20% | 28.7 kcal | 5.4 kcal |

| | | | |
|---|---|---|---|
| Weighted Average: | 31.4 kcal | Weighted error: | 3.1 kcal |

### DFT Calculations

All quantum chemical calculations were performed using the ORCA 4.1.0 software package. The geometries were calculated at the PBE0/Def2-SVP level of theory (frequency calculations at the same level were performed to validate each structure as a minimum or a transition state), followed by single-point calculations with a higher level of theory PBE0-D3/Def2-TZVP and SMD solvation model (N-Methylformamide as solvent) for more accurate energetic values. IRC (internal reaction coordinate) calculations starting from the transition structures were performed, and the reactants and products were verified.

### Results and Discussion

Heating a mixture of Nylon-6 and 5 mol % of La^{NTMS} to 240° C. for 4 h under 10-Torr vacuum afforded near quantitative polymer conversion to ε-caprolactam in 90% yield (Eq. 1, Table 1, entry 1). The product readily sublimed from the reaction hot zone and deposited as crystals on the cold reactor wall (Eq. 1). The reaction was rapid and reached near completion after 2 h (83% yield, Table 1, entry 2), with only slight increases of F-caprolactam yield upon further increasing the reaction time (3 h: 85% yield, Table 1, entry 3). Increasing the reaction temperature to 280° C. completed the reaction within 30 min, affecting neither caprolactam yield (93% yield, Table 1, entry 4) nor selectivity (>95%). Replacing the vacuum with an inert atmosphere did not significantly affect the reaction performance (83% vs. 85% yield, Table 1, entries 5 and 2, respectively). However, under vacuum, ε-caprolactam appeared more crystalline and collected further from the reaction hot zone. Without a catalyst, only minuscule amounts of caprolactam were obtained (Table 1, entry 6), along with trace impurities assignable to ε-caprolactam cyclic oligomers, which are common impurities in the industrial polymerization process. These oligomers can be extracted from the Nylon-6 starting material and observed by ¹H-NMR in CDCl₃.

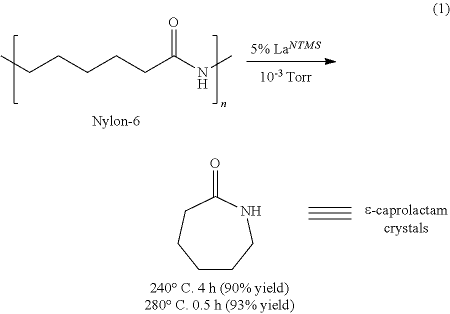

TABLE 1
Ligand and metal screening of Lanthanide
and Group 3 catalysts in Eq. 1.

| Entry | Catalyst | Reaction time | Yield^{[a]} |
|---|---|---|---|
| 1 | La^{NTMS} | 4 h | 90% |
| 2 | La^{NTMS} | 2 h | 83% |
| 3 | La^{NTMS} | 3 h | 85% |
| 4 ^{[b]} | La^{NTMS} | 30 min | 93% |
| 5 ^{[c]} | La^{NTMS} | 3 h | 83% |
| 6 | none | 3 h | 1.6% |
| 7 | La(NTf₂)₃ | 3 h | 7% |
| 8 | La(OTf)₃ | 3 h | 5% |
| 9 | La(OiPr)₃ | 3 h | 1.8% |
| 10 | LaCl₃ | 3 h | 1.4% |
| 11 | Nd^{NTMS} | 2 h | 60% |
| 12 | Sm^{NTMS} | 2 h | 55% |
| 13 | Gd^{NTMS} | 2 h | 48% |
| 14 | Lu^{NTMS} | 2 h | 45% |
| 15 | Y^{NTMS} | 2 h | 54% |
| 16 | Sc^{NTMS} | 2 h | 39% |

Conditions: 100 mg Nylon-6, 5 mol % catalyst loading, 50 mL Schlenk flask, neat, 240° C., 10⁻³ Torr.
^{[a]}Yield determined by ¹H-NMR using mesitylene as internal standard.
^{[b]} 280° C.
^{[c]} Glovebox atmosphere (1 atm Ar/N₂).

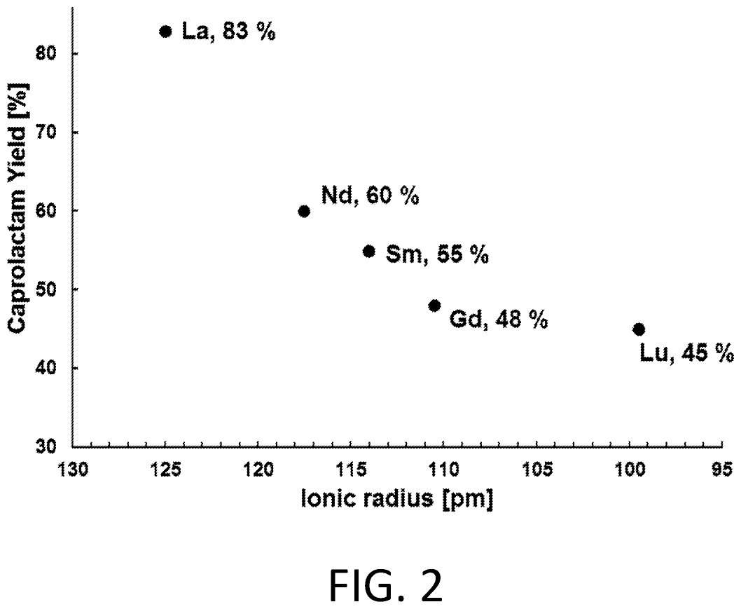

Exchanging the —N(TMS)₂ ligands for weakly basic triflate —OTf and bistriflimide —N(Tf)₂ ligands afforded poor catalytic activity (5% and 7% yield, Table 1, entries 7 and 8, respectively), highlighting that Lewis acidity alone cannot account for the observed activity. Furthermore, LaCl₃ and La(OⁱPr)₃ exhibited negligible catalytic activity (Table 1, entries 9 and 10), demonstrating the importance of the —N(TMS)₂ ligand in the catalysis, which was attributed to the high basicity in deprotonating the more acidic amide N—H bond (vide infra). Interestingly, screening different lanthanides revealed a clear trend of decreasing catalytic activity with decreasing ionic radius (FIG. 2). La^{NTMS} (Table 1, entry 2) was the most active complex, and the activity fell in the trend, Nd^{NTMS}>Sm^{NTMS}>Gd^{NTMS}>Lu^{NTMS} (Table 1, entries 11-14). The caprolactam yield after 2 h at 240° C. using the smallest lanthanide ion Lu^{NTMS} was 45% (Table 1, entry 14), vs. 83% for La^{NTMS} under identical conditions (Table 1, entry 2). Likewise, the catalytic activity of Y and Sc, the lighter and smaller lanthanum homologs, was tested. Following the trend of decreasing catalytic activity with ionic radius, Sc^{NTMS} (39% yield, Table 1, entry 16) was less active than Y^{NTMS} (54% yield, Table 1, entry 15), and both were less active than La^{NTMS} (83% yield, Table 1, entry 2). The lower activity for the smaller metal ions may reflect increased non-bonded repulsions in the sterically congested transition state of the rate-determining step, where the activated amide bond, the terminal amine, and two bulky —N(TMS)₂ ligands were all coordinated. This is also supported by DFT calculations (vide infra, FIGS. 3A-3C).

Next, a kinetic study was carried out to better understand the catalysis on a molecular level. Since La exhibited the highest activity in this study, La^{NTMS} was chosen for mechanistic investigations. Due to the lack of solvent and the viscous polymer reaction medium, classical kinetic studies in which the reaction progress is monitored in situ were not possible. Thus, a set of single-point experiments with varying reaction times and catalyst loadings were conducted to determine the rate law in substrate and catalyst concentrations.

Plotting the ε-caprolactam yield vs. time reveals a linear dependence (FIG. 3A), indicating that the reaction was zero-order in Nylon-6 under the present conditions, and suggesting that in the catalyst resting state, the polyamide was bound to a La center, probably after a Nylon amide (pKa=˜25.5)/bis(trimethylsilyl)amine (pKa=˜30) exchange. The La catalytic centers likely bind to the Nylon terminal amide moiety since, at this position, the free amine group at the chain end can also coordinate and stabilize it (FIG. 3B). This is supported by the DFT calculations (vide infra). Gel permeation chromatographic (GPC) analysis of the residual Nylon-6 solid at different reaction times revealed a gradual fall in the average molecular mass without significant changes in the dispersity, supporting a backbiting mechanism in which an ε-caprolactam molecule is eliminated from the polymer activated end in each catalytic cycle.

The dependence of the depolymerization yield on catalyst concentration reveals an interesting and informative picture (FIG. 3C). From 0 to 1.5 mol % catalyst loading, no activity was observed. This initial feature can be explained by catalyst deactivation involving the reactive carboxylic acid (pKa=5) chain ends, as shown in FIG. 3B. Indeed, in the case of 1 mol % La^{NTMS} loadings and Nylon 6 (Mₙ=11930 g/mol), the calculated carboxylic acid:La ratio is ˜1:1 assuming one carboxylic acid unit per 1 polymer chain. To test this hypothesis, a Nylon-6 sample was treated with 1 M aqueous KOH solution to deprotonate the acidic chain ends. After washing and drying, catalytic depolymerization experiments were conducted with the KOH-treated Nylon-6. Rapid quantitative depolymerization was observed with only 1 mol % La^{NTMS} (96% isolated yield, Table 2, entry 1), supporting a scenario in which the carboxylic acid groups deactivate a significant fraction of the catalytic units. Between 1.5 and 3 mol %, a steep near-linear activity increase was observed, implying the rate is first order in [catalyst]. Beyond 3 mol % catalyst, the system evidenced Michaelis-Menten-like saturation and even large increases in catalyst loading only marginally affected the reaction rate; in 45 min, 31% conversion for 5 mol % catalyst vs. 34% conversion for 11 mol % catalyst). Catalyst saturation would be achieved when every amide chain end was bound by a catalyst center and another step became turnover-limiting.

Thermogravimetric analysis (TGA) experiments were next conducted on Nylon-6+catalyst mixtures to estimate the experimental activation energy (data not shown). Isoconversional TGA is a standard method to investigate and extract kinetic data from complex solid-state systems, such as the pyrolysis of polymers or biomass. All sample manipulations were conducted in a glovebox to maintain an inert atmosphere. The observed TGA loss can be divided into three phases: 1) 25-270° C., mass loss due to TMS₂N— ligand protonolysis, 2) 270-350° C., catalyzed depolymerization, 3)>350° C., Nylon-6 pyrolysis. Four experiments at different heating rates were also conducted and used to estimate the activation energies at low conversions using the Flynn-Wall-Ozawa (FWO) method. The apparent depolymerization activation energy was Eₐ=31.4±3.1 kcal/mol.

Post-consumer waste plastic streams are generally composed of mixed plastics and polymer additives. To investigate the applicability of the La^{NTMS} catalyst to such scenarios, the depolymerization of Nylon-6 was carried out on 1:1 mixtures (by weight) with the common polymers PE, PP, and PET. PP and PE admixtures had no adverse effects on the depolymerization behavior, affording ε-caprolactam in 93% and 91% yield, respectively (Table 2, entries 2 and 3), while the unreacted PE or PP was recovered unchanged. The admixture of PET induced a slight decrease in catalytic activity, probably reflecting the presence of additional PET carboxylic acid or hydroxyl end groups (Table 2, entry 4). This process is also compatible with post-consumer Nylon-6 yarn, yielding ε-caprolactam in 78% yield (Table 2, entry 5).

TABLE 2
KOH-treated Nylon-6 depolymerization and the effect of
mixed plastics and post-consumer yarn on the catalysis.

| | | Catalyst | Reaction | |
|---|---|---|---|---|
| Entry | Polymers | loading | time | Yield ^{a} |
| 1 | KOH-treated Nylon-6 | 1% | 4 h | 97 (96) % |
| 2 | 1:1 mixture Nylon-6:PP | 5% | 6 h | 93% |
| 3 | 1:1 mixture Nylon-6:PE | 5% | 6 h | 91% |
| 4 | 1:1 mixture Nylon-6:PET | 5% | 6 h | 77% |
| 5^{b} | Post-consumer Nylon-6 | 4% | 24 h | 78% |

Conditions: 100 mg untreated Nylon-6, 50 mL Schlenk flask, solventless, 240° C., 10⁻³ Torr.
^{a} Yield determined by ¹H-NMR using mesitylene as an internal standard. Isolated yield is in parentheses.
^{b}Inert atmosphere instead of vacuum.

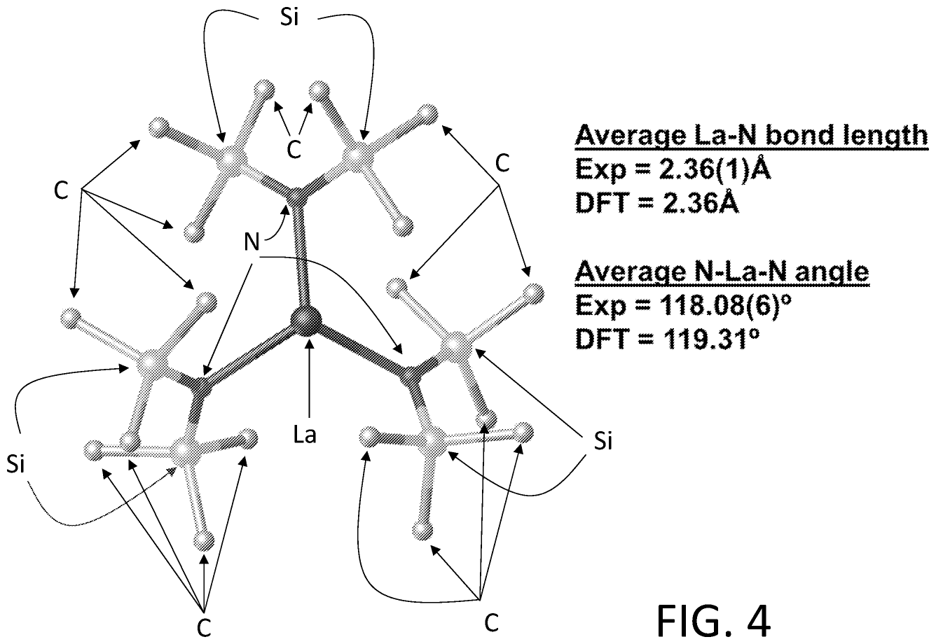

To further probe the Nylon-6 reaction mechanism and energetic landscape, a detailed solution-phase enthalpy energy profile was computed by DFT for the (TMS₂N)₃La (La^{NTMS}) catalyzed Nylon-6 depolymerization. First, the DFT-derived structure of La^{NTMS} was found to be in excellent agreement with the experimental X-ray structure, validating the computational approach (FIG. 4).

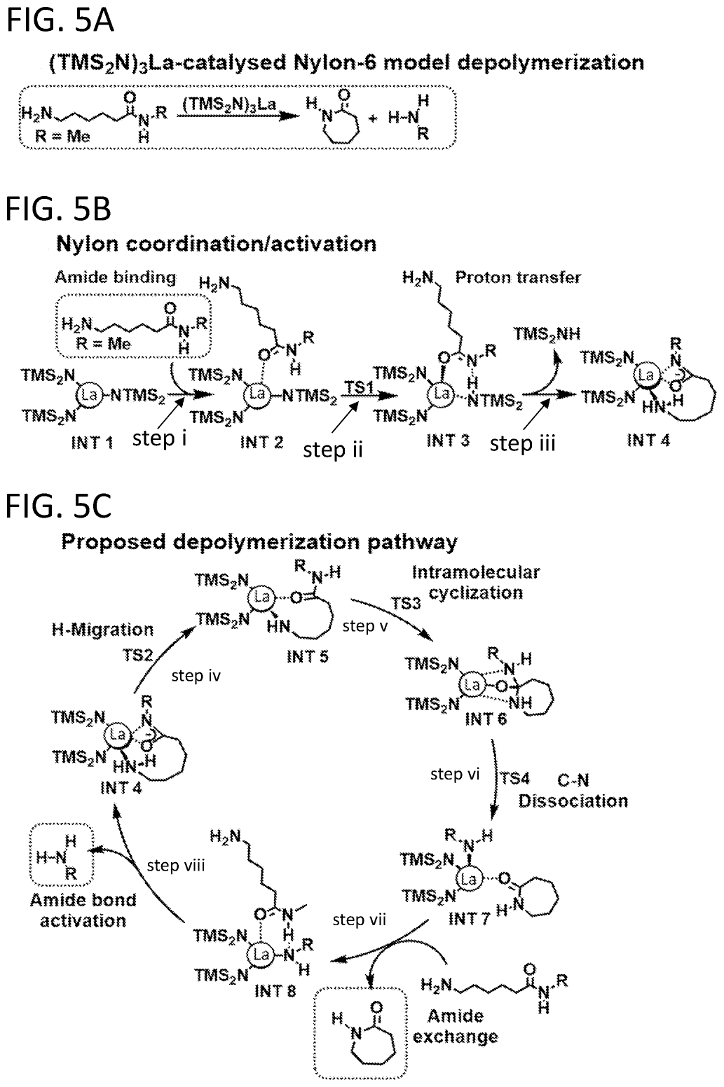

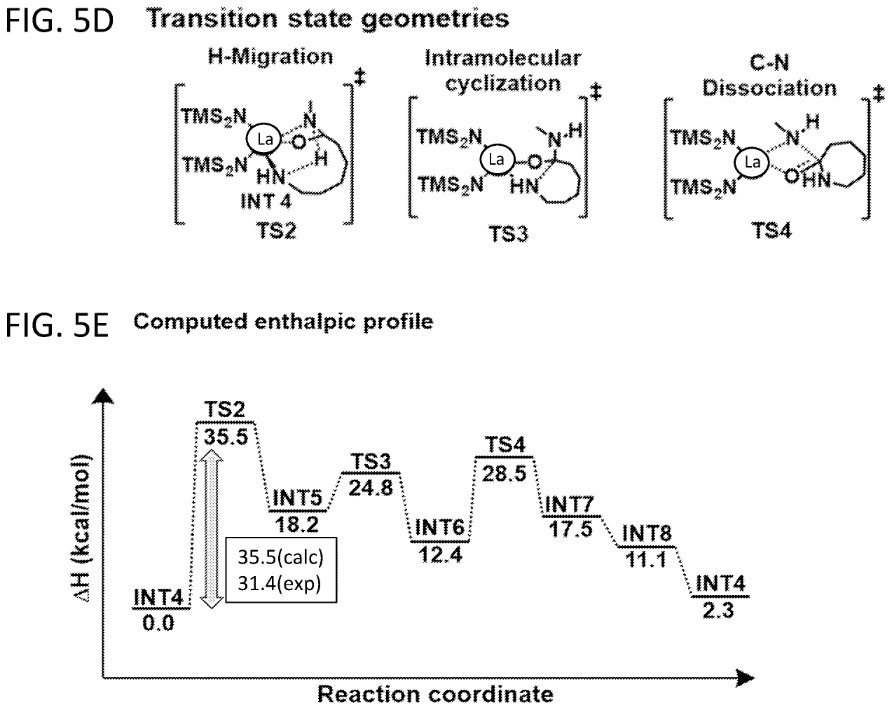

The secondary 6-amino-N-methylhexanamide was used as a Nylon-6 model for simplified computation (FIG. 5A). In the first step, the amide oxygen atom is coordinated to the Lewis acidic La₃₊ in (TMS₂N)₃La (INT1) to produce INT2 (FIG. 5B, step i). This step is highly exothermic (ΔH=−20.6 kcal/mol). Next, the amide proton migrates to the bis(silyl)amide ligand, yielding INT3. This step is endothermic by 5.0 kcal/mol with a low energetic barrier (TS1) of 5.4 kcal/mol (FIG. 5B, step ii). The exothermic elimination of TMS₂NH (ΔH=−2.4 kcal/mol) yields INT4 (FIG. 5B, step iii). This step is driven by the intramolecular coordination of the primary terminal amine, which stabilizes INT4 by 9.7 kcal/mol. Next, INT4 enters the catalytic cycle, and a proton transfer step from the primary amine to the amide moiety yields INT5 in an endothermic step (18.2 kcal/mol) (FIG. 5C, step iv). This step has a barrier of ΔH^{‡}=35.5 kcal/mol (TS2, FIG. 5D) and is the rate-determining transition state. Interestingly, when the primary terminal amine is replaced by a secondary amine, the barrier increases by 3.8 kcal/mol, highlighting once more the importance of the terminal NH₂ end group. Next, in an exothermic intramolecular cyclization (−5.8 kcal/mol), INT5 is converted to INT6 (FIG. 5C, step v). The cyclization step has a relatively low barrier of ΔH^{‡}=24.8 kcal/mol (TS3, FIG. 5D). A C—N bond dissociation step in INT6 yields INT7, which has the desired caprolactam product coordinated to the La center (FIG. 5C, step vi). This step is exothermic by 5.1 kcal/mol and has a barrier of 28.5 kcal/mol (TS4, FIG. 5D). Finally, caprolactam is spontaneously (−6.4 kcal/mol) released from INT7 when another secondary amide substrate coordinates to the La center yielding INT8 (FIG. 5C, step vii). Similar to steps ii and iii in FIG. 5B, amine (RNH₂) release from INT8 yields starting INT4 in an exothermic step (−8.8 kcal/mol). The overall computed reaction barrier is 35.5 kcal/mol, in good agreement with the TGA-derived Eₐ=31.4 kcal/mol. Moreover, the calculated reaction barrier for the less active Lu^{NTMS} catalyst is ˜1 kcal/mol higher than for La^{NTMS}, in good agreement with the experiment. As expected, the overall depolymerization reaction is endothermic (2.3 kcal/mol) (FIG. 5E). However, the constant caprolactam sublimation from the reaction mixture shifts the equilibrium towards the products.

### CONCLUSION

In conclusion, this Example demonstrated a catalytic system based on readily accessible lanthanide amides that catalyze the rapid and selective depolymerization of Nylon-6 to its industrial precursor, ε-caprolactam. The process was solvent-free, high-yielding, highly selective, and carried out under mild conditions. The catalytic activity correlated with the ionic radius of the lanthanide ion, with complexes bearing larger lanthanides demonstrating higher activities. The process was also compatible with post-consumer Nylon-6 and admixed plastics such as PP, PE, and PET. Experimental and theoretical mechanistic analyses establish that the reaction proceeded via a chain-end backbiting mechanism, where ε-caprolactam units were sequentially excised from the chain ends.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A method for depolymerizing a polyamide, the method comprising combining a polyamide and a lanthanide-organic catalyst comprising a lanthanide metal bound to at least one ligand, to depolymerize the polyamide to a product, wherein the at least one ligand is selected from those having a formula -EHₘ(XRₙ)₂ and benzyl,
wherein E is selected from N, C, P, Si, Ge, and Sn;
X is selected from Si H, N, P, C, Ge, and Sn;
R is selected from alkyl, H, aryl, alkoxyl, silyl, germyl, and stannyl;
wherein if E is N or P, then m=0 and if E is C, Si, Ge, or Sn, then m=1; and
further wherein n is from 2 to 3.

2. The method of claim 1, wherein the at least one ligand is selected from those having the formula -EHₘ(XRₙ)₂ and further wherein E is selected from N and C; X is selected from Si, and R is alkyl.

3. The method of claim 2, wherein the lanthanide metal is selected from La, Nd, Sm, Gd, Lu, Y, and Sc.

4. The method of claim 1, wherein all ligands bound to the lanthanide metal, including the at least one ligand, are of the same type.

5. The method of claim 4, wherein the at least one ligand is selected from those having the formula -EHₘ(XRₙ)₂ and further wherein E is selected from N and C; X is selected from Si, and R is alkyl.

6. The method of claim 5, wherein the lanthanide metal is selected from La, Nd, Sm, Gd, Lu, Y, and Sc.

7. The method of claim 1, wherein the lanthanide-organic catalyst is Ln[N(Si(CH₃)₃)₂]₃ or Ln[CH(Si(CH₃)₃)₂]₃, wherein Ln is the lanthanide metal.

8. The method of claim 7, wherein Ln is La, Nd, Sm, Gd, Lu, Y, or Sc.

9. The method of claim 8, wherein Ln is La or Nd.

10. The method of claim 1, wherein the lanthanide-organic catalyst is La[N(Si(CH₃)₃)₂]₃.

11. The method of claim 1, wherein the polyamide is a polymerization product of a monomer selected from 2-pyrrolidone, 2-piperidone, e-caprolactam, enantholactam, capryllactam, pelargolactam, azacycloundecan-2-one, azacyclotridecan-2-one, 6-azabicyclo[3.2.1]octan-7-one, and combinations thereof.

12. The method of claim 1, wherein the polyamide is selected from poly(2-pyrrolidinone) (Nylon-4), poly(2-piperidone) (Nylon-5), poly(hexano-6-lactam) (Nylon-6), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), poly[imino(1,6-dioxo hexamethylene)imino hexamethylene](Nylon-66), and combinations thereof.

13. The method of claim 1, wherein the product comprises a monomer from which the polyamide was formed.

14. The method of claim 13, wherein the monomer is a cyclic amide.

15. The method of claim 1, wherein the polyamide and the lanthanide-organic catalyst form a reaction mixture consisting of the polyamide and the lanthanide-organic catalyst.

16. The method of claim 1, wherein the method is carried out using a temperature of 250° C. or less and a loading of the lanthanide-organic catalyst of 5 mol % or less, and the method provides a yield of the product of at least 70%.

17. The method of claim 16, wherein the product comprises a monomer from which the polyamide was formed.

18. The method of claim 1, wherein the method is carried out using a continuous flow reactor system.

19. The method of claim 1, wherein the method further comprises recovering the product from a reaction mixture comprising the polyamide and the lanthanide-organic catalyst, wherein the product comprises a monomer from which the polyamide was formed.

20. The method of claim 1, wherein the lanthanide-organic catalyst is Ln[N(Si(CH₃)₃)₂]₃ or Ln[CH(Si(CH₃)₃)₂]₃, wherein Ln is La, Nd, Sm, Gd, Lu, Y, or Sc, and further wherein the method is carried out using a temperature of 250° C. or less and a loading of the lanthanide-organic catalyst of 5 mol % or less.

21. The method of claim 20, wherein the polyamide is poly(hexano-6-lactam) (Nylon-6), poly[imino(1,6-dioxo hexamethylene)imino hexamethylene](Nylon-66), or a combination thereof
